(19) United States
(12) Patent Application Publication
(10) Pub. No.: US 2018/0228992 A1
(43) Pub. Date: Aug. 16, 2018

(54) TUBE INTRODUCERS AND ASSEMBLIES

(71) Applicant: SMITHS MEDICAL INTERNATIONAL LIMITED, Ashford (GB)

(72) Inventors: Simon Neame, Broadstairs (GB); Eric Pagan, Hythe (GB)

(73) Assignee: SMITHS MEDICAL INTERNATIONAL LIMITED, Ashford (GB)

(21) Appl. No.: 15/736,137

(22) PCT Filed: Jun. 17, 2016
(86) PCT No.: PCT/GB2016/000121

§ 371 Date: Dec. 13, 2017

(30) Foreign Application Priority Data
Jul. 7, 2015 (GB) 1511881.3

(51) Int. Cl. A61M 16/04; A61M 25/06

(52) U.S. Cl. CPC A61M 16/0488; A61M 16/0418; A61M 16/0465; A61M 25/0668

(57) ABSTRACT

An introducer (2) for a curved tracheostomy tube (1) has a patient end tip (33) that projects beyond the patient end of the tube to help insertion. The patient end tip (33) is divided into two hinged portions (36) and (37). The hinged portions (36) and (37) bend outwardly to fill the gap created when the tube 1 is deformed to an oval section caused by straightening it by loading on the introducer.

This invention relates to tube introducers of the kind adapted for insertion within the bore of a medico-surgical tube, the introducer having a forward, patient end tip adapted to project from the patient end of the tube.

In various medical and surgical procedures it is necessary to introduce a plastics tube into the body through a natural or surgically-created opening. These tubes can be rigid or flexible although it is often desirable for the tube to be flexible, in order to conform to the anatomy of a body passage or to reduce trauma to patient tissue. Both rigid and flexible tubes require some form of handle at their patient end to help direct and apply the forces needed for insertion. Flexible tubes often need to be supported internally by a stiffer introducer or obturator that can be removed once the tube has been inserted to the desired position. One application for such introducers is to introduce a tracheostomy tube into a tracheostomy. The introducer has a tapered tip that projects beyond the patient end of the tube to help guide the assembly of introducer and tube through a surgically-made passage through tracheal tissue. In order to facilitate a smooth, atraumatic insertion with a low insertion force it is desirable for there to be a smooth transition between the surface of the tip of the introducer and the patient end tip of the tube. One problem with assemblies of tracheostomy tubes and introducers is that, when the tube is straightened from its normal angled shape for insertion the cross section of the tube at the patient end tip is distorted from a circular to an oval shape. This causes a gap to be formed between the circular surface of the introducer and the oval tip of the tube. This gap can increase the resistance to inserting the assembly into the trachea and can increase the damage caused to tracheal tissue during insertion. EP1099451 describes an obturator for a tracheostomy tube having a collapsible tip that expands to fill a distorted oval shape at the patient end of a straightened tracheostomy tube.

It is an object of the present invention to provide an alternative tube introducer and an assembly of an introducer and tube.

According to one aspect of the present invention there is provided a tube introducer of the above-specified kind, characterised in that the patient end tip is divided along a part of its length into two or more hinged portions joined together towards their patient end, and that the patient end tip is arranged such that the two portions can be compressed inwardly towards one another and are resiliently expansible outwardly.

The introducer preferably has a passage along its length, the passage being provided along a major part of the length of the introducer by an open channel. The hinged portions are preferably arranged to be expansible in the plane of curvature of the tube. The hinged portions are preferably tapered along a major part of their length from the forward end such that the effective diameter of the patient end tip increases rearwardly, and a shorter part of the length of the hinged portions may taper in the opposite sense to a smaller diameter rearwardly. The forward, patient end tip may have a passage extending through it for receiving a guide member, the passage through the patient end tip having a ramp formation such that when the guide member is inserted it engages the ramp formation and urges the hinged portions outwardly away from one another. The introducer preferably includes a retainer arranged to retain the introducer with a rear end of the tube. The retainer preferably includes a catch arranged to engage a part of a connector at the machine end of the tube.

According to another aspect of the present invention there is provided an assembly of a medico-surgical tube having a curve along a part at least of its length and an introducer according to the above one aspect of the present invention, wherein the introducer extends along the tube and straightens the tube, and wherein the patient end of the introducer projects from the patient end of the tube.

The tube is preferably a tracheostomy tube.

An introducer and assembly according to the present invention will now be described, by way of example, with reference to the accompanying drawings, in which:

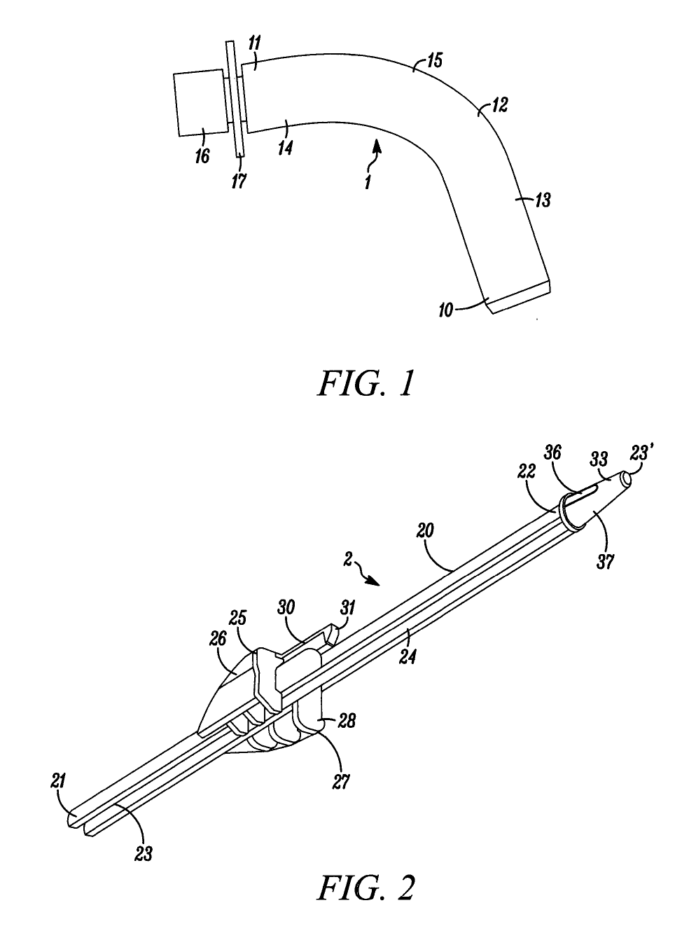

FIG. 1 is a side elevation view of a tracheostomy tube before the introducer is inserted;

FIG. 2 is a perspective view of the introducer;

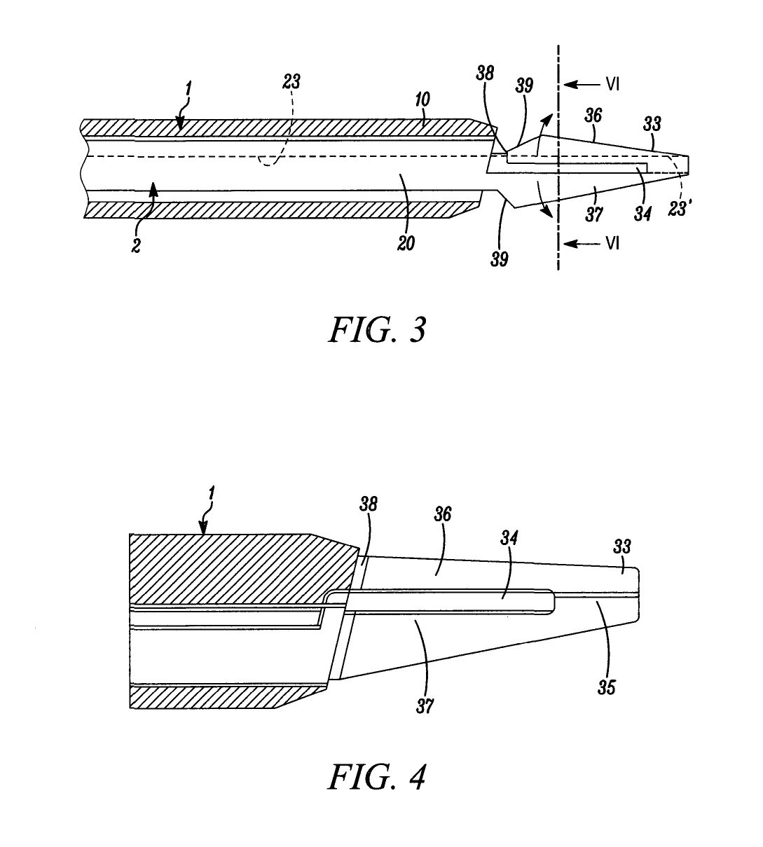

FIG. 3 is an enlarged part cross-sectional side elevation view of the patient end of the assembly;

FIG. 4 is an enlarged side elevation view of the patient end of the assembly;

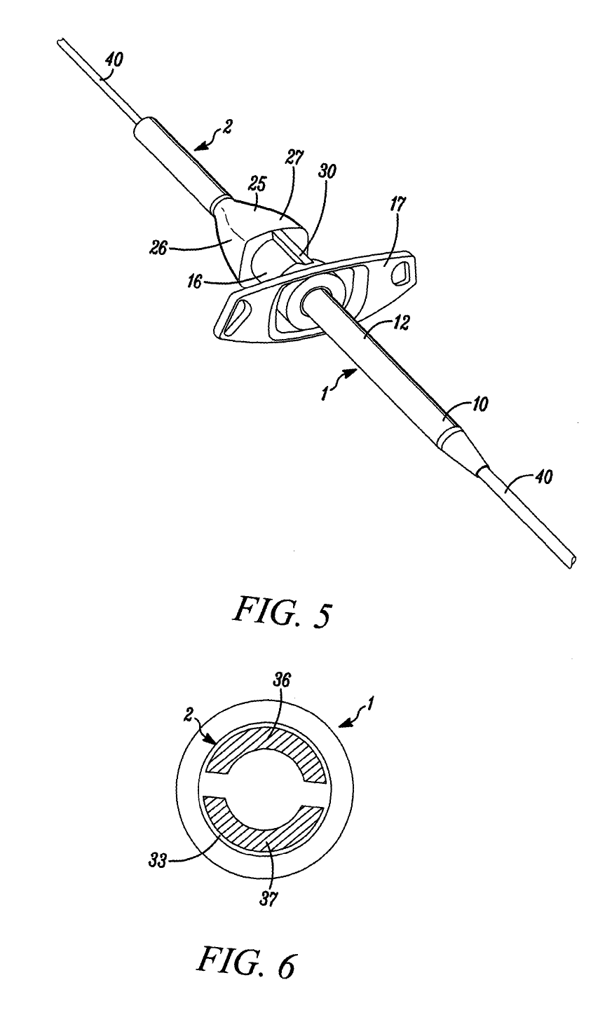

FIG. 5 is a perspective view of the assembly of the tracheostomy tube and introducer; and

FIG. 6 is a transverse cross-sectional view along the line VI-VI of FIG. 3.

With reference to FIG. 1 there is shown a conventional tracheostomy tube 1 with a patient end 10 adapted to be located within the trachea and a machine end 11 adapted lie project externally from the neck surface. The shaft 12 of the tube 1 may be of various shapes but, in this example, comprises a straight patient end portion 13, a straight machine end portion 14 and a curved intermediate portion 15. The machine end 11 of the tube 1 has a conventional 15 mm female connector 16 by which connection is made to the tube, and a flange 17 to which a neck strap (not shown) can be attached in order to secure the tube about the patient's neck. The tube 1 could be provided with a conventional inflatable sealing cuff close to its patient end but is shown as being without any such cuff.

With reference now to FIGS. 2 to 6 there is shown an introducer 2 for use in inserting the tracheostomy tube 1 through a surgically made opening into the trachea. The introducer 2 has a shaft 20 extending along its length from its rear, machine end 21 to its forward, patient end 22. The shaft 20 is of a plastics material, is straight and of channel shape having a passage 23 extending along its length that is open along its lower surface 24. The shaft 20 is sufficiently stiff to be able to retain the tube 1 in a straight configuration when this is loaded on the introducer 2, as shown in FIG. 5.

At a location about one third the way along the shaft 20 from its rear, machine end 21 the introducer 2 has an enlarged retainer formation 25 of substantially square section and tapering height and width along its length, being smaller towards its rear end than its forward end. The retainer 25 has two opposite side faces 26 and 27 that are ribbed to improve grip and that are angled outwardly towards the patient end of the introducer. The front face 28 of the retainer 25 is flat and provides a stop against which the rear end of the tracheostomy tube 1 abuts during use. The front face 28 also supports a retaining clip 30 projecting forwardly from one edge and having an inwardly-extending catch 31 at one end positioned to engage the forward edge of the connector 16 on the tube 1. The retainer 25 serves two purposes, one being to provide a grip for the finger and thumb of the clinician, with the rear part of the shaft 20 extending across his palm for support. The other purpose is to retain the machine end 11 of the tube 1 on the introducer 2 against both forward and rearward forces so that it can be inserted into the trachea or pulled out of the trachea, such as for repositioning, should this be necessary. The retaining clip 30 is easily released when necessary, either by lifting its free end manually or simply by twisting the introducer 2 relative to the tube 1.

The introducer 2 is completed, at its forward or patient end 22 by a nose or patient end tip portion 33, which tapers along its length to a smaller size at its patient end. The nose 33 has, in its natural state, an asymmetric, oval cross section, at least at its rear end. The shape and size of the oval is selected to match as closely as possible the oval shape of the bore of the tracheostomy tube 1 at its forward end when the tube is straightened by loading onto the introducer 2. More particularly, the major axis of the oval is aligned with the normal plane of curvature of the tracheostomy tube 1 and its minor axis is aligned orthogonally to this. The nose 33 has two slots 34 extending longitudinally diametrically opposite one another in a plane including the minor axis of the oval shape. The slots 34 extend from the rear end of the nose 33 but are spaced from the patient end of the nose by a region 35 so that the upper and lower parts of the nose remain attached or hinged by this region. The slots 34 divide the rear part of the nose 33 into an upper and lower limb or hinged portion 36 and 37 that can be flexed or hinged resiliently towards and away from each other. The lower limb 37 is attached with the shaft 20 whereas the upper limb 36 is not attached with the shaft directly but is free moving at its rear end 38. The forward, patient end of both limbs 36 and 37 tapers rearwardly so that the effective diameter of the nose increases rearwardly along most of its length but the shorter, rear end of the nose 33 is formed with a reverse taper 39, that is, it reduces in diameter rearwardly. This reverse taper 39 ensures that the nose 33 can be pulled back into the tube 1 when the introducer 2 is withdrawn. The construction of the nose 33 enables its rear end 38 to be compressed radially inwardly when it is inserted through the connector 16 and the rear end of the tube 1, which retain a circular section, but the two limbs 36 and 37 are then free to expand outwardly up and down to fill the deformed oval shape at the patient end 10 of the tube 1 produced by straightening the tube on the introducer. The bore 23′ through the nose 33 could have a ramp profile (not shown) on one or both limbs 36 or 37, such that, when a guide member such as a guiding catheter or guidewire 40 (FIG. 5) is extended through the channel 23 along the shaft 20 it engages the or each ramp and displaces the or each limb outwardly. When the guide member 40 is withdrawn the material memory of the nose portion 33 returns it to its original shape. The time for which the guide member 40 is used is insufficient for the material of the nose portion 33 to adopt a prolonged set with the limbs 36 and 37 deformed outwardly.

The slots 34 could be filled by a material that is displaced or folded by the action of inserting the guide member so as to reduce the risk that the slots could snag on patient tissue when the assembly is inserted. Instead of having two limbs, the patient end of the introducer could be divided into three or more hinged limbs.

The invention is not confined to use with tracheostomy tubes but could be used with other tubes for insertion in a body cavity. Although the introducer described above is straight it could be curved (that is, continuously curved along its length) or arced (that is, with two straight regions separated by a curved region) although the advantage of the present invention arises when the tube with which is used is distorted in cross-section when straightened for insertion.

1-9. (canceled)

10. A tube introducer adapted for insertion within the bore of a medico-surgical tube, the introducer having a forward, patient end tip adapted to project from the patient end of the tube, characterised in that the patient end tip is divided along a part of its length into two or more hinged portions joined together towards their patient end, and that the patient end tip is arranged such that the two portions can be compressed inwardly towards one another and are resiliently expansible outwardly.

11. A tube introducer according to claim 10, characterised in that the introducer has a passage along its length, and that the passage is provided along a major part of the length of the introducer by an open channel.

12. A tube introducer according to claim 10, characterised in that the hinged portions are arranged to be expansible in the plane of curvature of the tube.

13. A tube introducer according to claim 10, characterised in that the hinged portions are tapered along a major part of their length from the forward end such that the effective diameter of the patient end tip increases rearwardly, and that a shorter part of the length of the hinged portions tapers in the opposite sense to a smaller diameter rearwardly.

14. A tube introducer according claim 10, characterised in that the forward, patient end tip has a passage extending through it for receiving a guide member, and that the passage through the patient end tip has a ramp formation such that when the guide member is inserted it engages the ramp formation and urges the hinged portions outwardly away from one another.

15. A tube introducer according to claim 10, characterised in that the introducer includes a retainer arranged to retain the introducer with a rear end of the tube.

16. A tube introducer according to claim 15, characterised in that the retainer includes a catch arranged to engage a part of a connector at the machine end of the tube.

17. An assembly of a medico-surgical tube having a curve along a part at least of its length and an introducer adapted for insertion within the bore of the tube, the introducer having a forward, patient end tip adapted to project from the patient end of the tube, wherein the patient end tip is divided along a part of its length into two or more hinged portions joined together towards their patient end and is arranged such that the two portions can be compressed inwardly towards one another and are resiliently expansible outwardly, wherein the introducer extends along the tube and straightens the tube, and wherein the patient end of the introducer projects from the patient end of the tube.

18. An assembly according to claim 17, characterised in that the tube is a tracheostomy tube.
